(19) United States
(12) Patent Application Publication
(10) Pub. No.: US 2002/0051767 A1
(43) Pub. Date: May 2, 2002

(54) CANCER TREATMENT

(76) Inventors: YAWEN L. CHIANG, POTOMAC, MD (US); ESTHER H. CHANG, REDWOOD CITY, CA (US)

Correspondence Address: RAYMOND J LILLIE, CARELLA BYRNE BAIN GILFILLAN, CECCHI STEWART & OLSTEIN, 6 BECKER FARM ROAD, ROSELAND, NJ, 07068

(21) Appl. No.: 08/527,373

(22) Filed: Sep. 13, 1995

(51) Int. Cl. A61K 48/00; C07H 21/04; A61K 39/145

(52) U.S. Cl. 424/93.21; 514/44; 514/2; 424/209.1; 435/320.1; 435/91.1; 435/91.31; 435/252.3; 536/24.1

(57) ABSTRACT

A process for improving the treatment of a tumor by radiation therapy which comprises treating a tumor by radiation therapy wherein the cells of the tumor have been transduced with a polynucleotide encoding wild-type p53, such as, for example, by transducing the tumor cells with an adenoviral vector including a DNA sequence encoding wild-type p53. Such a combination treatment of the transduction of tumor cells with a polynucleotide encoding wild-type p53 and radiation therapy provides a more effective treatment than by using p53 gene therapy alone or radiation therapy alone.

This invention relates to cancer treatment and more particularly to the treatment of cancer by gene therapy. Still more particularly, the invention relates to treatment of cancer by a combination of gene therapy and radiation therapy.

### BACKGROUND OF THE INVENTION

Cancer is a devastating disease, and there is a need to find new methods for treatment, particularly for those cancers which have been particularly difficult to treat.

For example, each year in the U.S. approximately 40,000 individuals will be diagnosed with squamous cell carcinoma of the head and neck (SCCHN) and upper aerodigestive tract. This disease not only has a profound effect upon speech, swallowing and physical appearance, but has an overall survival rate of only approximately 50%, a rate which has remained relatively unchanged for more than thirty years. Treatment often requires aggressive adjunctive therapy after surgery, with radiation being the most common form. However, a significant number (30-40%) of squamous cell carcinomas of the head and neck have been found to be resistant to radiotherapy. Failure to respond to radiation therapy has been an unmet medical need in the treatment of head and neck tumors and other forms of cancer as well. The identification of an improved therapy would have immense clinical significance.

Squamous cell carcinomas of the head and neck area (SCCHN) arise from a multiplicity of sites and are primarily due to environmental factors, principally, the use of alcohol and tobacco.

These tumors are notoriously difficult to treat with a high percentage of recurrence. One of the major problems is the presence of microscopic residual tumor after surgery at the site of the primary tumor. Despite advances in current treatments, patients with advanced disease have a poor prognosis. More than two thirds of the individuals in whom the diagnosis of squamous cell carcinoma of the head and neck is made will present with state III or IV disease at the primary and/or nodal sites. Despite optimal local therapy, 50%-60% of these patients will ultimately develop local recurrence, and 30% or more will develop distant metastatic disease. The overall survival rate is 40% for patients whose tumors are completely resected and 20% for those with unresectable tumors treated with radiotherapy alone. Treatment of recurrent disease is palliative at best and without long-term benefit. Furthermore, the long-term outlook for those surviving their first malignancy is overshadowed by a 10% to 40% rate of a second primary malignancy.

Incorporation of chemotherapy into the early treatment regimen of locally advanced disease has reduced morbidity by increasing organ preservation. However, as the majority of those receiving chemotherapy still develop recurrence of the primary disease, its role in increasing overall survival remains to be demonstrated. Historically, surgery has been the primary form of treatment, with the addition of radiotherapy in advanced stages in an effort to reduce recurrence. Recently, combined modality approaches employing chemotherapy, joint chemoradiotherapy or alternating chemo and radio therapies have been tried. However, only a few of the studies, those using simultaneous chemo-radio treatment, were able to demonstrate increased survival. Stupp, et al., Seminars in Oncology, Vol. 21, pgs. 349-358 (1994).

Since the early part of the century radiation has been the treatment of choice for most head and neck cancers and remains an integral part, either as a single modality or as a part of a multimodal course, of treatment for this disease. The most commonly used radioisotopes employed in radiotherapy are X and gamma rays (photon radiation). Additionally, charged (electrons, protons, and heavy ions such as helium and neon) and uncharged particles (neutrons) can also be used in radiation treatment.

Standard radiation therapy in the U.S. consists of five daily (fractionated) doses of 1.8 to 2.25 Gy per week given continuously for five to seven weeks. The total dose is dependent upon the size of the tumor, its histology and the normal tissue tolerance. Gross visible SCCHN tumors usually require total doses of 65 to 75 Gy while microscopic disease usually receives 45 to 50 Gy over 4½ to 5½ weeks. The extent of treatment is also dependent on the site and stage of the disease (reviewed in Awan, et al., Hematology/Oncology Clinics of North America, Vol. 5, pgs. 635-655 (1991)). Several non-standard patterns of treatment have also been examined (Awan, et al., 1991): 1) Hyperfractionation, where smaller doses are given more than once per day over the same time span as with standard treatment; 2) Accelerated Fractionation, where multiple daily doses are given resulting in shortening of overall treatment time; 3) Split-course, where standard doses are given but with a break midway in the treatment course; and 4) Hypofractionation, which is usually one or two large doses given weekly for several weeks. Many of the non-standard regimens being employed may be a combination of one or more of the four described above. (Awan, et al., 1991).

Many factors contribute to the control of head and neck cancer. Besides the growth characteristics and the number of cells in the tumor, various aspects of the tumor microenvironment, including the pH, reoxygenation, and hypoxia are important. Also to be considered is the inherent radioresistance/sensitivity of the tumor cells themselves. However, the genetic basis of ionizing radiation resistance (RR) in mammalian cells is poorly understood. The individual molecular events and specific genes involved will affect both normal cellular protection from radiation damage as well as failure of tumors to respond to radiation therapy.

P53 may also play a role in the development and progression of SCCHN. Depending upon the tissue source and method of detection of abnormal p53, both the gene and its expression have been identified in 33% to 100% of head and neck cancers. Mutations have been found in exons 4 through 9 with a hot spot in the codon 238-248 region (Field, et al., Arch. Otolaryngol. Head and Neck Surg., Vol. 119, pgs. 1118-1122 (1993); Brachman, Seminars in Oncology, Vol. 21, pgs. 320-329 (1994)) Moreover, preliminary studies indicate that the presence of p53 mutations may be indicative of a higher frequency and shorter median time to recurrence of the tumor (Brachman, 1994) Abnormal p53 also correlates with a history of heavy smoking and drinking which are the primary environmental factors associated with SCCHN (Field, et al., 1993)

Although, Kastan, et al., Cancer Research, Vol. 51, pgs. 6304-6311 (December 1, 1991), discloses that p53 expression increases after treatment of ML-1 myeloid leukemia cells with gamma-radiation, whereby such cells remain in G₁ arrest, Jung, et al., Cancer Research, Vol. 52, pgs. 6390-6393 (1992) disclose mutations in the p53 gene in the SCC-35, JSQ-3, SQ-38, SCC-9, and SCC-9G squamous carcinoma cell lines. The SCC-35 and JSQ-3 cell lines are radiation resistant, while the SQ-38, SCC-9 and SCC-9G cell lines are radiation sensitive.

### SUMMARY OF THE INVENTION

The present invention is directed to the treatment of tumors whereby such tumors are treated with a combination of radiation therapy and transduction with a polynucleotide encoding wild type p53. Such treatment may be employed in treating radiation resistant tumors as well as radiation-sensitive tumors.

### BRIEF DESCRIPTION OF THE DRAWINGS

The invention now will be described with respect to the drawings, wherein:

FIG. 1 is a map of plasmid pAvS6;

FIG. 2 is a map of plasmid pAvS6-nLacZ;

FIG. 3 is a schematic of the construction of Av1LacZ4;

FIG. 4 is a schematic of the construction of pAvS6.p53;

FIG. 5 is a schematic of the construction of Av1p53;

FIG. 6 is a graph of the D₁₀ values (Gy) of JSQ-3 cells at 24 and 36 hours after treatment with 5, 10, or 20 MOI of Av1p53 or 20 MOI of Av1LacZ4;

FIG. 7 is a graph of the mean tumor volumes in mice injected with JSQ-3 cells followed by radiation treatment and treatment with 10 MOI Av1p53 or 10 MOI Av1LacZ4; and

FIG. 8 is a graph of the mean tumor volumes in mice injected with JSQ-3 cells followed by no treatment, radiation treatment alone, Av1p53 treatment alone, or radiation treatment combined with treatment with Av1p53 or Av1LacZ4.

### DETAILED DESCRIPTION OF THE INVENTION

In accordance with one aspect of the present invention, there is provided a process for treating cancer, and in particular squamous cell carcinoma of the head and neck and upper aerodigestive tract, by a combination of gene therapy and radiation therapy.

More particularly, the present invention is directed to cancer treatment wherein tumor cells are transduced or transfected with a polynucleotide encoding wild type p53 protein in conjunction with irradiation of the tumor.

Applicants have found that such combination results in a more effective treatment than using p53 gene therapy alone or radiation therapy alone. Applicants also have found that such treatment enhances the radiation sensitivity of tumor cells, whether such tumor cells normally are radiation resistant or radiation sensitive. Applicants have found that the transduction of radiation resistant tumor cells with a polynucleotide encoding wild type p53 can reverse the radiation resistance of such tumor cells. Thus, the treatment of the present invention may be employed in treating radiation resistant tumors as well as radiation sensitive tumors.

Thus, in accordance with an aspect of the present invention, there is provided a process for treating radiation resistant tumors wherein the effect of radiation therapy is enhanced by providing cells of the tumor with a polynucleotide encoding wild type p53.

The term “treating a tumor” as used herein means that one provides for the inhibition, prevention, or destruction of the growth of the tumor cells.

The term “polynucleotide” as used herein means a polymeric form of nucleotide of any length, and includes ribonucleotides and deoxyribonucleotides. Such term also includes single- and double-stranded DNA, as well as single- and double-stranded RNA. The term also includes modified polynucleotides such as methylated or capped polynucleotides.

The gene encoding wild-type p53 is obtainable through sources known to those skilled in the art (e.g., Genbank, ATCC, etc.), and/or may be isolated from expression vehicles (e.g., plasmids) obtainable through sources known to those skilled in the art through standard techniques (e.g., PCR) known to those skilled in the art.

The polynucleotide encoding wild-type p53 may be contained within an appropriate expression vehicle which has been transduced into the cell. Such expression vehicles include, but are not limited to, plasmids, eukaryotic vectors, prokaryotic vectors (such as, for example, bacterial vectors), and viral vectors.

In one embodiment, the vector is a viral vector. Viral a vectors which may be employed include RNA virus vectors (such as retroviral vectors), and DNA virus vectors (such as adenoviral vectors, adeno-associated virus vectors, Herpes Virus vectors, and vaccinia virus vectors). When an RNA virus vector is employed, in constructing the vector, the polynucleotide encoding wild-type p53 is in the form of RNA. When a DNA virus vector is employed, in constructing the vector, the polynucleotide encoding wild-type p53 is in the form of DNA.

In a preferred embodiment, the viral vector is an adenoviral vector.

The adenoviral vector which is employed may, in one embodiment, be an adenoviral vector which includes essentially the complete adenoviral genome (Shenk et al., Curr. Top. Microbiol. Immunol., 111(3): 1-39 (1984). Alternatively, the adenoviral vector may be a modified adenoviral vector in which at least a portion of the adenoviral genome has been deleted.

In the preferred embodiment, the adenoviral vector comprises an adenoviral 5′ ITR; an adenoviral 3′ ITR; an adenoviral encapsidation signal; a DNA sequence encoding wild-type p53; and a promoter controlling the DNA sequence encoding wild-type p53. The vector is free of at least the majority of adenoviral E1 and E3 DNA sequences, but is not free of all of the E2 and E4 DNA sequences, and DNA sequences encoding adenoviral proteins promoted by the adenoviral major late promoter.

In one embodiment, the vector also is free of at least a portion of at least one DNA sequence selected from the group consisting of the E2 and E4 DNA sequences.

In another embodiment, the vector is free of at least the majority of the adenoviral E1 and E3 DNA sequences, and is free of a portion of the other of the E2 and E4 DNA sequences.

In still another embodiment, the gene in the E2a region that encodes the 72 kilodalton binding protein is mutated to produce a temperature sensitive protein that is active at 32° C., the temperature at which the viral particles are produced. This temperature sensitive mutant is described in Ensinger et al., J. Virology, 10:328-339 (1972), Van der Vliet et al., J. Virology, 15:348-354 (1975), and Friefeld et al., Virology, 124:380-389 (1983).

Such a vector, in a preferred embodiment, is constructed first by constructing, according to standard techniques, a shuttle plasmid which contains, beginning at the 5′ end, the “critical left end elements,” which include an adenoviral 5′ ITR, an adenoviral encapsidation signal, and an E1a enhancer sequence; a promoter (which may be an adenoviral promoter or a foreign promoter); a multiple cloning site (which may be as herein described); a poly A signal; and a DNA segment which corresponds to a segment of the adenoviral genome. The vector also may contain a tripartite leader sequence. The DNA segment corresponding to the adenoviral genome serves as a substrate for homologous recombination with a modified or mutated adenovirus, and such sequence may encompass, for example, a segment of the adenovirus 5 genome no longer than from base 3329 to base 6246 of the genome. The plasmid may also include a selectable marker and an origin of replication. The origin of replication may be a bacterial origin of replication. Representative examples of such shuttle plasmids include pAvS6, which is described in published PCT Application Nos. WO94/23582, published Oct. 27, 1994, and WO95/09654, published Apr. 13, 1995. The DNA sequence encoding wild-type p53 may then be inserted into the multiple cloning site to produce a plasmid vector.

This construct is then used to produce an adenoviral vector. Homologous recombination is effected with a modified or mutated adenovirus in which at least the majority of the E1 and E3 adenoviral DNA sequences have been deleted. Such homologous recombination may be effected through co-transfection of the plasmid vector and the modified adenovirus into a helper cell line, such as 293 cells, by CaPO₄ precipitation. Upon such homologous recombination, a recombinant adenoviral vector is formed that includes DNA sequences derived from the shuttle plasmid between the Not I site and the homologous recombination fragment, and DNA derived from the E1 and E3 deleted adenovirus between the homologous recombination fragment and the 3′ lTR.

In one embodiment, the homologous recombination fragment overlaps with nucleotides 3329 to 6246 of the adenovirus 5 (ATCC VR-5) genome.

Through such homologous recombination, a vector is formed which includes an adenoviral 5′ ITR, an adenoviral encapsidation signal; an E1a enhancer sequence; a promoter; a DNA sequence encoding wild-type p53 protein; a poly A signal; adenoviral DNA free of at least the majority of the E1 and E3 adenoviral DNA sequences; and an adenoviral 3′ ITR. The vector also may include a tripartite leader sequence. The vector may then be transfected into a helper cell line, such as the 293 helper cell line (ATCC No. CRL1573), which will include the E1a and E1b DNA sequences, which are necessary for viral replication, and to generate adenoviral particles. Transfection may take place by electroporation, calcium phosphate precipitation, microinjection, or through proteoliposomes.

The vector hereinabove described may include a multiple cloning site to facilitate the insertion of the DNA sequence encoding the wild-type p53 into the cloning vector. In general, the multiple cloning site includes “rare” restriction enzyme sites; i.e., sites which are found in eukaryotic genes at a frequency of from about one in every 10,000 to about one in every 100,000 base pairs. An appropriate vector is thus formed by cutting the cloning vector by standard techniques at appropriate restriction sites in the multiple cloning site, and then ligating the DNA sequence encoding wild-type p53 into the cloning vector.

The DNA sequence encoding wild-type p53 is under the control of a suitable promoter, which may be selected from those herein described, or such DNA may be under the control of its own native promoter.

In one embodiment, the adenovirus may be constructed by using a yeast artificial chromosome (or YAC) containing an adenoviral genome according to the method described in Ketner, et al., PNAS, Vol. 91, pgs. 6186-6190 (1994), in conjunction with the teachings contained herein. In this embodiment, the adenovirus yeast artificial chromosome is produced by homologous recombination in vivo between adenoviral DNA and yeast artificial chromosome plasmiid vectors carrying segments of the adenoviral left and right genomic termini. A DNA sequence encoding wild-type p53 then may be cloned into the adenoviral DNA. The modified adenoviral genome then is excised from the adenovirus yeast artificial chromosome in order to be used to generate adenoviral vector particles as hereinabove described.

The adenoviral vector particles are administered to an animal host in an amount which in combination with radiation therapy is effective to inhibit, prevent, or destroy the growth of the tumor cells. Such animal hosts include mammalian hosts, including human and non-human primate hosts. The adenoviral vector particles may be administered systemically, such as, for example, by intravenous, intraarterial, or intraperitoneal administration. Alternatively, the adenoviral vector particles may be administered by direct, nonsystemic injection of the adenoviral vector particles to site of the tumor. In general, the adenoviral vector particles are administered at a multiplicity of infection of from about 5 to about 20. The exact dosage of adenoviral vector particles which is to be administered is dependent upon a variety of factors, including the age, weight, and sex of the patient, and the type and severity of the tumor to be treated.

The adenoviral particles may be administered as part of a preparation containing adenoviral particles in an amount of at least 1×10⁷ pfu, and in general not exceeding 1×10¹⁰ pfu preferably from about 5×10⁷ pfu to about 1×10⁹ pfu, and more preferably from about 5×10⁷ pfu to about 5×10⁸ pfu. The adenoviral particles may be administered in combination with a pharmaceutically acceptable carrier in a volume up to 100 ml.

The adenoviral vector particles may be administered in combination with a pharmaceutically acceptable carrier suitable for administration to a patient, such as, for example, a liquid carrier such as a saline solution, protamine sulfate (Elkins-Sinn, Inc., Cherry Hill, N.J.), water, aqueous buffers such as phosphate buffers and Tris buffers, or Polybrene (Sigma Chemical, St. Louis, Mo.). The selection of a suitable pharmaceutical carrier is deemed to be apparent to those skilled in the art from the teachings contained herein.

In another embodiment, the viral vector is a retroviral vector. Examples of retroviral vectors which may be employed include, but are not limited to, Moloney Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus. The vector is generally a replication incompetent retrovirus particle.

Retroviral vectors are useful as agents to mediate retroviral-mediated gene transfer into eukaryotic cells. Retroviral vectors are generally constructed such that the majority of sequences coding for the structural genes of the virus are deleted and replaced by the gene(s) of interest. Most often, the structural genes (i.e., gag, pol, and env), are removed from the retroviral backbone using genetic engineering techniques known in the art. This may include digestion with the appropriate restriction endonuclease or, in some instances, with Bal 31 exonuclease to generate fragments containing appropriate portions of the packaging signal.

These new genes have been incorporated into the proviral backbone in several general ways. The most straightforward constructions are ones in which the structural genes of the retrovirus are replaced by a single gene which there is transcribed under the control of the viral regulatory sequences within the long terminal repeat (LTR). Retroviral vectors have also been constructed which can introduce more than one gene into target cells. Usually, in such vectors one gene is under the regulatory control of the viral LTR, while the second gene is expressed either off a spliced message or is under the regulation of its own, internal promoter. Alternatively, two genes may be expressed from a single promoter by the use of an Internal Ribosome Entry Site.

Efforts have been directed at minimizing the viral component of the viral backbone, largely in an effort to reduce the chance for recombination between the vector and the packaging-defective helper virus within packaging cells. A packaging-defective helper virus is necessary to provide the structural genes of a retrovirus, which have been deleted from the vector itself.

Examples of retroviral vectors which may be employed include, but are not limited to, Moloney Murine Leukemia Virus vectors such as those described in Miller, et al., Biotechniques, Vol. 7, pgs. 980-990 (1989), and in Miller, et al., Human Gene Therapy, Vol. 1, pgs. 5-14 (1990).

In a preferred embodiment, the retroviral vector may include at least four cloning, or restriction enzyme recognition sites, wherein at least two of the sites have an average frequency of appearance in eukaryotic genes of less than once in 10,000 base pairs; i.e., the restriction product has an average DNA size of at least 10,000 base pairs. Preferred cloning sites are selected from the group consisting of NotI, SnaBI, SalI, and XhoI. In a preferred embodiment, the retroviral vector includes each of these cloning sites. Such vectors are further described in U.S. patent application Ser. No. 08/340,805, filed Nov. 17, 1994, and in PCT Application No. WO91/10728, published Jul. 25, 1991, and incorporated herein by reference in their entireties.

When a retroviral vector including such cloning sites is employed, there may also be provided a shuttle cloning vector which includes at least two cloning sites which are compatible with at least two cloning sites selected from the group consisting of NotI, SnaBI, SalI, and XhoI located on the retroviral vector. The shuttle cloning vector also includes at least one desired gene which is capable of being transferred from the shuttle cloning vector to the retroviral vector.

The shuttle cloning vector may be constructed from a basic “backbone” vector or fragment to which are ligated one or more linkers which include cloning or restriction enzyme recognition sites. Included in the cloning sites are the compatible, or complementary cloning sites hereinabove described. Genes and/or promoters having ends corresponding to the restriction sites of the shuttle vector may be ligated into the shuttle vector through techniques known in the art.

The shuttle cloning vector can be employed to amplify DNA sequences in prokaryotic systems. The shuttle cloning vector may be prepared from plasmids generally used in prokaryotic systems and in particular in bacteria. Thus, for example, the shuttle cloning vector may be derived from be plasmids such as pBR322; pUC 18; etc.

The vector includes one or more promoters. Suitable promoters which may be employed include, but are not limited to, the retroviral LTR; the SV40 promoter; and the human cytomegalovirus (CMV) promoter described in Miller, et al., Biotechniques, Vol. 7, No. 9, 980-990 (1989), or any other promoter (e.g., cellular promoters such as eukaryotic cellular promoters including, but not limited to, the histone, pol III, and β-actin promoters). Other viral promoters which may be employed include, but are not limited to, adenovirus promoters, TK promoters, and B19 parvoviruis promoters. The selection of a suitable promoter will be apparent to those skilled in the art from the teachings contained herein.

The vector then is employed to transduce a packaging cell line to form a producer cell line. Examples of packaging cells which may be transfected include, but are not limited to, the PE501, PA317, ψ-2, ψ-AM, PA12, T19-14X, VT-19-17-H2, ψ CRE, ψ CRIP, GP+E-86, GP+envAm12, and DAN cell lines, as described in Miller, Human Gene Therapy, Vol. 1, pgs. 5-14 (1990). The vector containing the polynucleotide encoding wild-type p53 transduces the packaging cells through any means known in the art. Such means include, but are not limited to, electroporation, the use of liposomes, and CaPO₄ precipitation.

The packaging cells thus become producer cells which generate retroviral vectors which include a polynucleotide encoding wild-type p53. Such retroviral vectors then are transduced into the tumor cells, whereby the transduced tumor cells will express p53.

The retroviral vectors are administered to a host in an amount which in combination with radiation therapy is effective to inhibit, prevent, or destroy the growth of the tumor cells. Such administration may be by systemic administration as hereinabove described, or by direct injection of the retroviral vectors in the tumor. In general, the retroviral vectors are administered in an amount of at least 1×10⁷ cfu, and in general, such an amount does not exceed 1×10⁸ cfu. Preferably, the retroviral vectors are administered in an amount of from about 2×10⁷ cfu to about 5×10⁷ cfu. The exact dosage to be administered is dependent upon a variety of factors including those hereinabove described.

The retroviral vectors also may be administered in conjunction with an acceptable pharmaceutical carrier, which may be as hereinabove described.

In another alternative, retroviral producer cells, such as those derived from the packaging cell lines hereinabove described, which include a polynucleotide encoding wild type p53, may be administered to a host. Such producer cells may, in one embodiment, be administered systemically (e.g., intravenously or intraarterially) at a point in close proximity to the tumor, or the producer cells may be administered directly to the tumor. The producer cell line then produces retroviral vectors including a polynucleotide encoding wild type p53 in vivo, whereby such retroviral vectors then transduce the tumor cells.

In conjunction with the transduction of the tumor cells with a polynucleotide encoding wild-type p53, radiation also is administered to the tumor cells in an amount effective to inhibit, prevent, or destroy the growth of the tumor cells.

Radiation which may be employed includes, but is not limited to, X-rays, and gamma-rays (photon radiation) Also, charged (e.g., electrons, protons, and heavy ions such as helium and neon) and uncharged particles (e.g., neutrons) may be employed in radiation treatment. In general, the radiation is administered at about the same time or subsequent to the transduction of the tumor cells with a polynucleotide encoding wild-type p53. The radiation may be administered as a single dose or in multiple doses administered at intervals of from about 24 hours to about 48 hours. The total dose of radiation administered may be from about 20 Gy to about 50 Gy, preferably from about 20 Gy to about 25 Gy. Preferably, the radiation is administered in 10 doses in an amount of from about 2.0 Gy to about 2.5 Gy per dose. The exact dosage of radiation to be administered is dependent upon a variety of factors including those hereinabove described, as well as the normal tissue tolerance of the area exposed to the radiation.

Tumors which may be treated in accordance with she present invention include malignant and non-malignant tumors. The tumors may be those which normally are radiation resistant, as well as non-radiation resistant (i.e., radiation-sensitive) tumors.

Malignant (including primary and metastatic) tumors which may be treated include, but are not limited to, cancers which may be found in the oral epithelium, including, but not limited to, squamous cell carcinomas of the mouth, oral cavity, and upper aerodigestive tract, including the floor of the mouth, tongue, cheek, gums, or palate, adenocarcinoma of the oral cavity, lip cancers, Kaposi's sarcoma, and laryngeal papillomas and nasopharyngeal cancers which may have spread to the oral epithelium; tumors occurring in the adrenal glands; bladder, bone; breast; cervix; endocrine glands (including thyroid glands, the pituitary gland, and the pancreas); stomach; small intestine; peritoneal cavity; colon; rectum; heart; hematopoietic tissue; kidney; liver; lung; muscle; nervous system; brain; eye; oral cavity; pharynx; larynx and other head and neck cancers; ovaries; penis; prostate; skin (including melanoma, basal cell carcinoma, and squamous cell carcinoma); testicles; thymus; and uterus.

The present invention is applicable particularly to the treatment of squamous cell carcinomas of the head and neck, and of the upper aerodigestive tract. For example, in one embodiment, a viral vector, such as an adenoviral vector, is administered to an animal host in an amount of from about 5×10⁷ pfu to about 5×10⁸ pfu. At about 36 hours to about 48 hours after injection of the viral vectors, radiation is administered in 8 to 10 doses in an amount of from about 2.0 Gy to about 2.5 Gy per dose.

### EXAMPLES

The invention now will be described with respect to the following examples; however, the scope of the present invention is not intended to be limited thereby.

### Materials

A. In Vitro System. The cell line employed is the SCCHN cell line JSQ-3. This line was derived from a tumor of the nasal vestibule which had failed radiotherapy (Weichselbaum, et al., Int. J. Radiation Oncology Biol. Phys., Vol. 15, pgs. 575-579 (1988)). This cell line is characterized as being significantly radiation resistant with a D, value of 263 (Weichselbaum, et al., Head and Neck Cancer, Vol. 3, pgs 399-407 (1993)) More recently, it was shown by Jung, et al., 1992, to carry a mutated form of p53. Two mutations were identified in the cell line, one in exon 4 at codon 72 (G-->C) and one in exon 8 at codon 298 (G-->T) (Jung, et al., 1992). In addition, an activated form of the raf-1 gene was isolated from this cell line (Kasid, et al., Science, Vol. 237, pgs. 1039-1041 (1987)). These cells, therefore, represent a good model to test the effectiveness of the replacement of wt p53 (via an adenoviral vector) on decreasing radiation resistance.

B. In Vivo Model. A nude mouse model system is employed to determine if the introduction of replicative defective human adenovirus carrying wt p53 (Av1p53) is effective in inhibiting the growth of a xenograft induced by cells derived from a squamous cell carcinoma of the human head and neck (JSQ-3), both alone and in combination with radiation treatment. The model is similar to that described by Clayman et al., Cancer Research, Vol. 55, pgs. 1-6 (1995). In this xenograft model tumor cells are injected subcutaneously on the lower back of the mouse. This system is advantageous in that this system facilitates exposure of the resulting tumor to radiation. Moreover, it is easy to measure tumor size and therefore to assess the effectiveness of the treatment. This model can also be used to mimic the post-surgical microscopic residual environment of SCCHN. With this model, the major difference from the examples contained herein is that in the examples contained herein the emphasis is placed on the combination of Av1p53 and radiotherapy.

C. Construction of Av1LacZ4 and Av1p53

The adenoviral vectors Av1LacZ4 and Av1p53 are replication deficient E1a/E1b, E3 deletion mutants containing a LacZ (β-galactosidase) gene and a p53 gene, respectively.

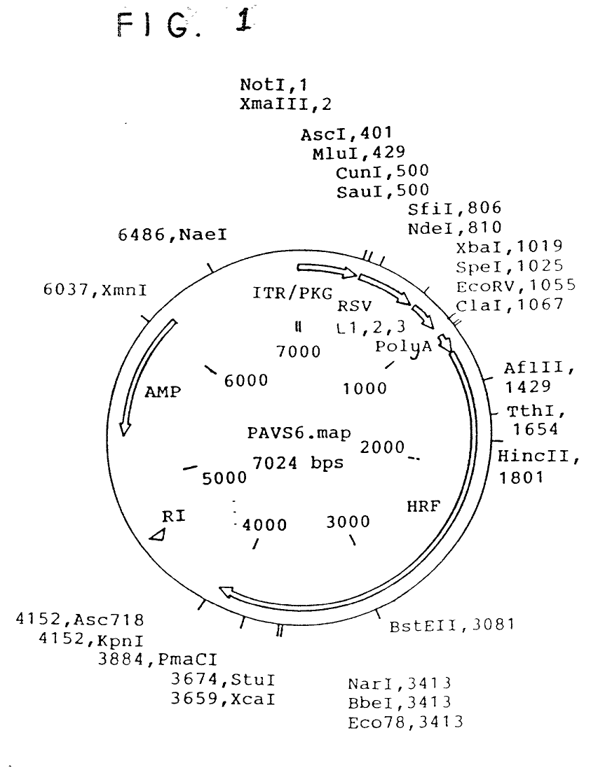

Av1LacZ4 was constructed from the adenoviral shuttle vector pAvS6 (FIG. 1), which is described in published PCT Application Nos. WO94/23582, published Oct. 27, 1994 and WO95/09654, published Apr. 13, 1995.

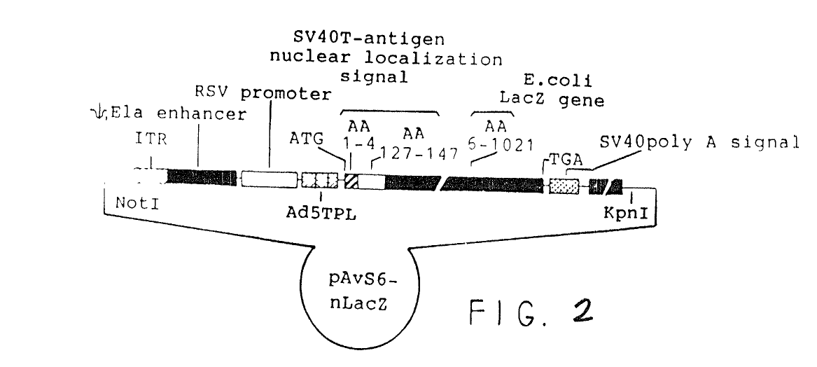

The recombinant, replication-deficient adenoviral vector Av1LacZ4, which expresses a nuclear-targetable B-galactosidase enzyme, was constructed in two steps. First, a transcriptional unit consisting of DNA encoding amino acids 1 through 4 of the SV40 T-antigen followed by DNA encoding amino acids 127 through 147 of the SV40 T-antigen (containing the nuclear targeting peptide Pro-Lys-Lys-Lys-Arg-Lys-Val), followed by DNA encoding amino acids 6 through 1021 of E. coli B-galactosidase, was constructed using routine cloning and PCR techniques and placed into the EcoRV site of pAvS6 to yield pAvS6-nlacZ (FIG. 2).

The infectious, replication-deficient, Av1LacZ4 was assembled in 293 cells by homologous recombination. To accomplish this, plasmid pAvS6-nLacZ was linearized by cleavage with KpnI. Genomic adenoviral DNA was isolated from purified Ad-dl327 viruses by Hirt extraction, cleaved with ClaI, and the large (approximately 35 kb) fragment was isolated by agarose gel electrophoresis and purified. Ad-dl327 (Thimmapaya, et al., Cell, Vol. 31, pg. 543 (1983)) is identical to Adenovirus 5 except that an XbaI fragment including bases 28591 to 30474 (or map units 78.5 to 84.7) of the Adenovirus 5 genome, and which is located in the E3 region, has been deleted. The ClaI fragment was used as the backbone for all first generation adenoviral vectors, and the vectors derived from it are known as Avl.

Five micrograms of linearized plasmid DNA (pAvS6n-LacZ) and 2.5 μg of the large ClaI fragment of Ad-dl327 then were mixed and co-transfected into a dish of 293 cells by the calcium phosphate precipitation method. After 16 hours, the cells were overlaid with a 1:1 mixture of 2% Sea Plaque agar and 2x medium and incubated in a humidified, 37° C., 5% CO₂/air environment until plaques appeared (approximately one to two weeks). Plaques were selected and intracellular vector was released into the medium by three cycles of freezing and thawing. The lysate was cleared of cellular debris by centrifugation. The plaque (in 300 μl) was used for a first round of infection of 293 cells, vector release, and clarification as follows:

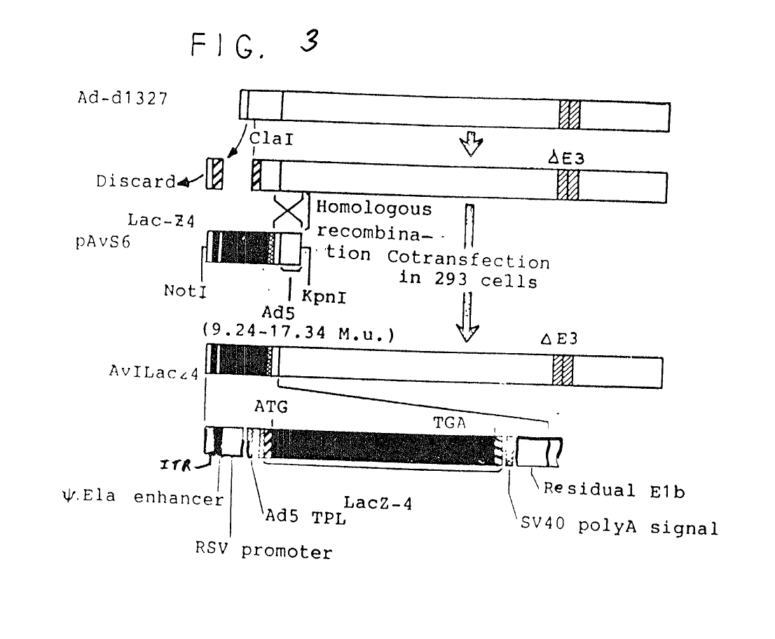

One 35 mm dish of 293 cells was infected with 100 μl of plaque lysate plus 400 μl of IMEM-2 (IMEM plus 2% FBS, 2 mM glutamine (Bio Whittaker 046764)) plus 1.5 ml of IMEM-10 (Improved minimal essential medium (Eagle's) with 2x glutamine plus 10% vol./vol. fetal bovine serum) plus 2 mM supplemental glutamine (Bio Whittaker 08063A) and incubated at 37° C. for approximately three days until the cytopathic effect, a rounded appearance and “grapelike” clusters, was observed. Cells and supernatant were collected and designated as CVL-A. Av1LacZ4 vector (a schematic of the construction of which is shown in FIG. 3) was released by three cycles of freezing and thawing of the CVL A. Then, a 60 mm dish of 293 cells was infected with 0.5 ml of the CVL-A plus 3 ml of IMEM-10 and incubated for approximately three days as above. Cells and supernatant from this infection then were processed by three freeze/thaw cycles in the same manner. Av1LacZ4 also is described in Yei, et al., Human Gene Therapy, Vol. 5, pgs. 731-744 (1994); Trapnell, Advanced Drug Delivery Reviews, Vol. 12, pgs. 185-199 (1993), and Smith, et al., Nature Genetics, Vol. 5, pgs. 397-402 (December 1993), which are incorporated herein by reference.

The resultant viral stock was titered by plaque assay on 293 cells using a standard protocol involving a 1.5 hour adsorption period in DMEM/2% FBS, followed by washout and agar overlay of the cell monolayer. (Graham, et al., Virology, Vol. 52, pgs. 456-467 (1973)). The absence of wild-type virus was checked by polymerase chain reaction assays of the stock using primers amplifying a 337 bp fragment of the E1 gene. The stock was negative for wild-type adenovirus using this assay.

The virus stock then was frozen at −80° C. and stored until used. The virus stock had a titer of 1.5×10¹¹ pfu/ml.

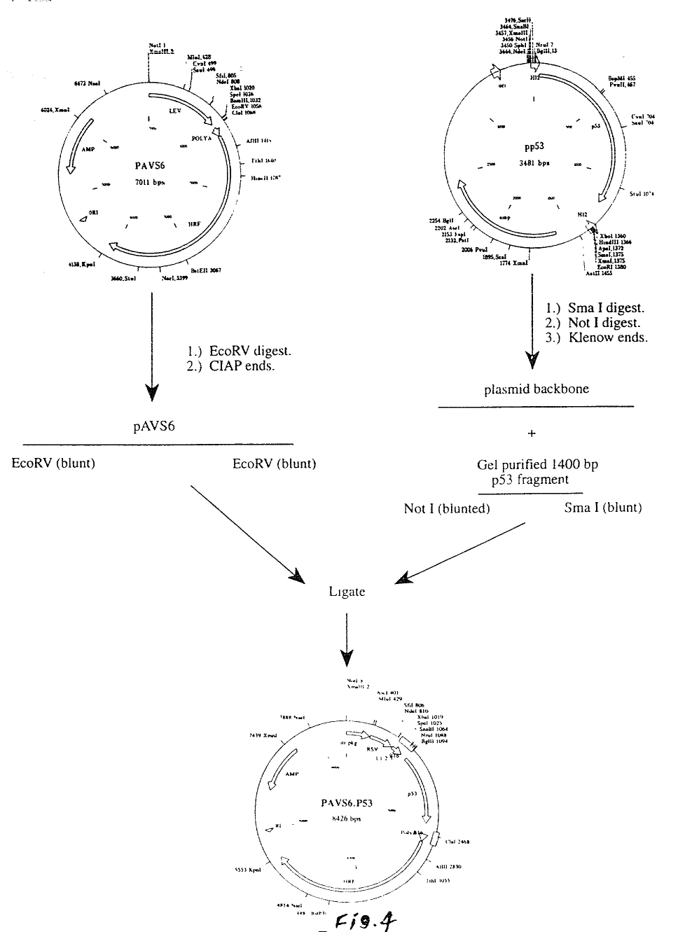

Av1p53 was generated from the plasmid nAvS6.p53 (FIG. 4), which was constructed from pAvS6. pAvS6 was digested with EcoRV, and the ends were blunted with calf intestinal alkaline phosphatase. The EcoRV digest linearized the plasmid, opening it in the region between the tripartite leader sequence and the poly A sequence. The linear pAvS6 fragment was gel purified. The p53 gene was obtained from plasmid pp53 (FIG. 4). pp53 was derived from the shuttle plasmid pBg(described in published PCT Application No. WO91/10728, published Jul. 25, 1991), whereby the β-galactosidase gene of pBg is removed and replaced with the p53 gene. The plasmid pp53 is digested with SmaI and NotI, and a resulting 1.4 kb fragment including the p53 gene is blunt ended at the 5′ end with Klenow (The 3′ end was blunt ended as a result of the SmaI digest.) and gel purified. The 1.4 kb NotI-SmaI fragment including the p53 gene is ligated to the EcoRV fragment obtained from pAvS6 to generate pAvS6.p53 (FIG. 4).

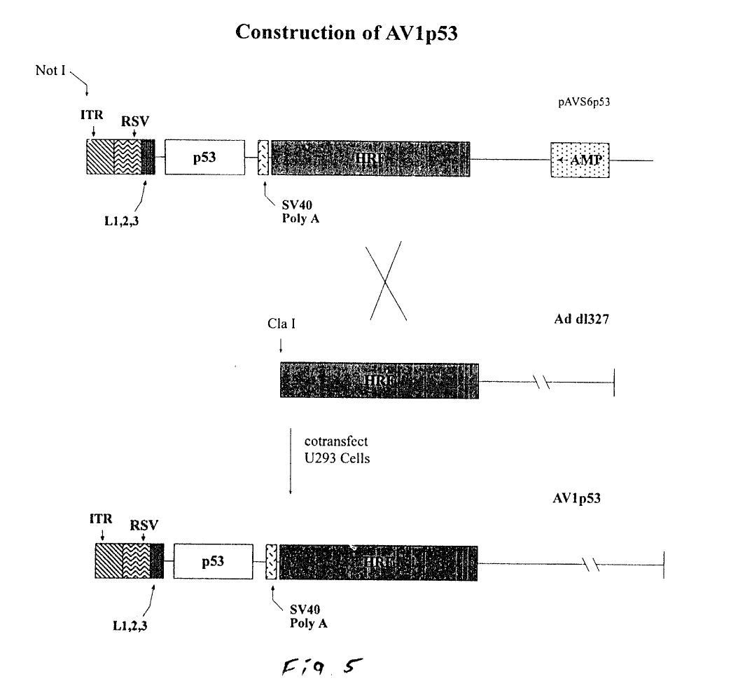

Av1p53 (FIG. 5) then is generated from pAvS6.p53 by linearization of pAvS6.p53 with NotI, followed by homologous recombination with the large ClaI fragment of Ad-dl327 according to the same procedure as hereinabove described with respect to the generation of Av1LacZ4 from pAvS6n-LacZ. Virus was isolated from plaques formed in the 293 cell monolayer. The presence of the p53 gene was confirmed by Southern Blot analysis.

### Example 1

3×10⁴ JSQ-3 cells were plated per well in a 24 well tissue culture dish. 24 hours later at approximately 50%. confluency, they were treated with Av1p53 or Av1LacZ4 in doses of 0 (control), 10, 20, 40, 80, 160, or 320 MOI. Treatment consisted of incubating the cells with the appropriate virus concentration in a volume of 150 μl of PBS for two hours at 37° C. with gentle rocking. At the end of the two hours, 2 ml of media (McCoy's 5A with 10% FBS) was added to the wells without removing the virus. 48 hours after the first virus treatment, a second virus dose was given in the same manner. 4 days after the second virus treatment the cells were fixed and stained with Giemsa Stain. Visual observation of the status of the cells was made with regard to morphology to approximate the amount of cell killing. The visual estimations of the percentage inhibition of the growth of the JSQ-3 cells treated with Av1p53 were as follows:

| | |
|---|---|
| 0 MOI | 0% |
| 10 MOI | 10% |
| 20 MOI | 30% |
| 40 MOI | 50% |
| 80 MOI | 90% |
| 160 MOI | 100% |

The visual estimations of the percentage inhibition of the growth of the JSQ-3 cells treated with Av1LacZ4 were as follows:

| | |
|---|---|
| 0 MOI | 0% |
| 10 MOI | 0% |
| 20 MOI | 0% |
| 40 MOI | 0% |
| 80 MOI | 10% |
| 160 MOI | 30% |
| 320 MOI | 40% |

### Example 2

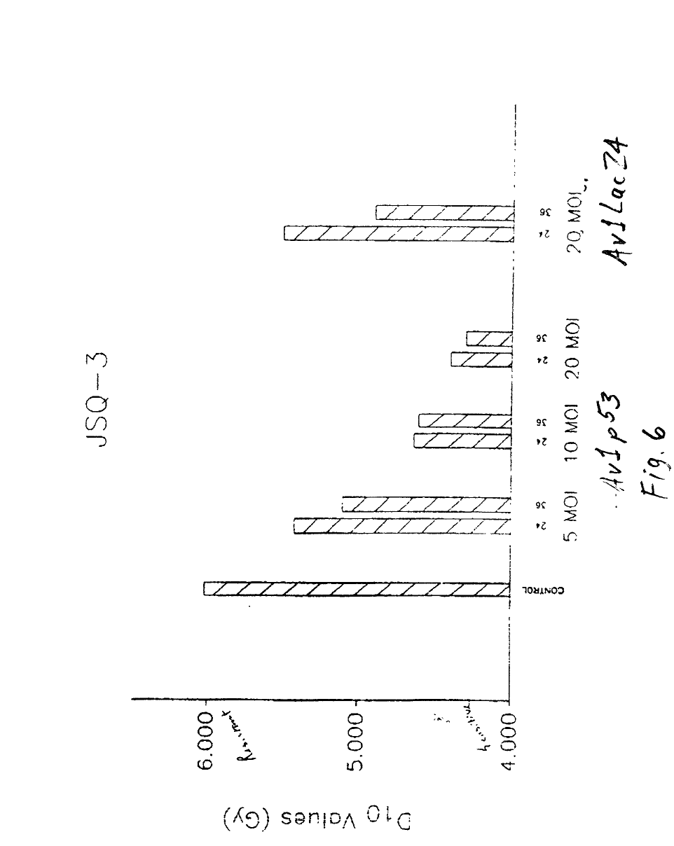

In this experiment, 1.2×10⁵ JSQ-3 cells were plated in each well of a 6 well tissue culture dish. 24 hours later they were treated with 5, 10, or 20 MOI of Av1p53 or 20 MOI of Av1LacZ4 in 500 μl of PBS. 24 or 36 hours after virus treatment the cells were trypsinized, counted, exposed to various doses of gamma radiation and replated. 10-14 days later, the plates were fixed and stained with Giemsa stain. Colonies of 50 or more normal looking cells were counted and the percent survival for each radiation dose calculated (number of colonies/cells plated) . D₁₀ is the radiation dose in Grays (Gy) required to reduce survival to 10%. A D₁₀ value of 6.0 Gy indicates significant radiation resistance. Normal radiation sensitive cells usually have D₁₀ values of between 3 and 4 Gy. The data, as shown in FIG. 6, clearly shows a dose response relationship with both increasing doses of Av1p53 and time. The Av1p53 treated cells are significantly more radiation sensitive than the untreated controls. With 20 MOI the D₁₀ value is reduced from 6.02 Gy to 4.3 Gy, a value much closer to that of non-radioresistant cells. Conversely, treatment with 20 MOI of Av1LacZ4 has a minimal effect on the survival level of the cells.

These results clearly demonstrated that the restoration of wt p53 is capable of verting the radiation resistant phenotype of this SCCHN cell line in vitro.

### Example 3

4-6 week old female athymic nude mice were injected subcutaneously on the lower back above the tail with 1×10⁶ JSQ-3 cells (a radiation resistant head and neck tumor cell line) in 50 ul PBS. Eight days later tumors of approximately 2×3×1 mm were evident at the injection site. The animals were then divided into three groups, and injected, directly into the tumor, with either 5 MOI (5×10⁷ pfu) or 10 MOI (1×10⁸ pfu) of Av1p53 (in 50 ul PBS) or 10 MOI (1×10⁸ pfu) of the control LacZ vector (Av1LacZ4) in 50 ul PBS. 36-48 hours post injection the tumor area only was exposed to a 1.5 Gy dose of X-rays. The same day a second injection of either Av1p53 or Av1LacZ4 was administered. 36-48 hours later, the tumor site was exposed to a 2.0 Gy dose of radiation. Thereafter, the animals were given radiation every 48 hours to a total dose of 20 Gy. As a control, one mouse, which had been treated with 10 MOI of Av1p53, received no radiation.

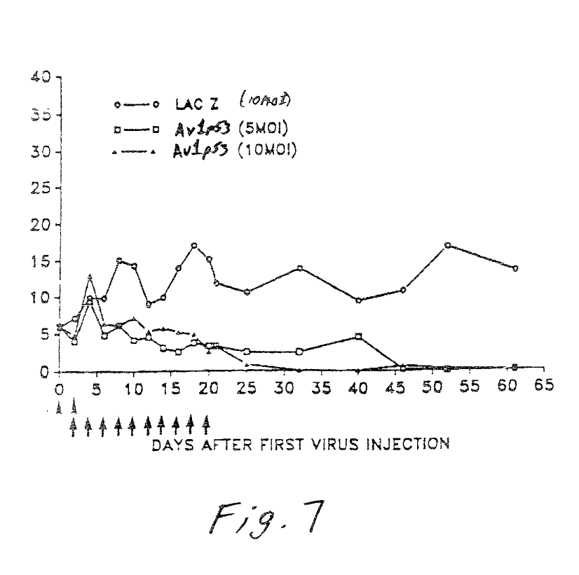

The size of each tumor was measured prior to each radiation treatment and the mean tumor volume plotted against time. At the start of the experiment, the Av1p53 5 MOI group contained 5 animals, the Av1p53 10 MOI group contained 6 animals, and the Av1LacZ4 group had 4 animals. By the end of the experiment, due to death and sacrificing of animals for histology, there were 3, 5, and 3 animals surviving, respectively. As shown in FIG. 7, the top two arrows indicate virus injections, while the bottom arrows represent doses of radiation. This experiment indicates that Av1p53, even at 5 MOI, in combination with radiation, was able to inhibit and decrease tumor growth, even 40 days after the end of the radiation treatments. On the other hand, the Av1LacZ4 treated tumors, even in the presence of radiation, did not decrease in size.

### Example 4

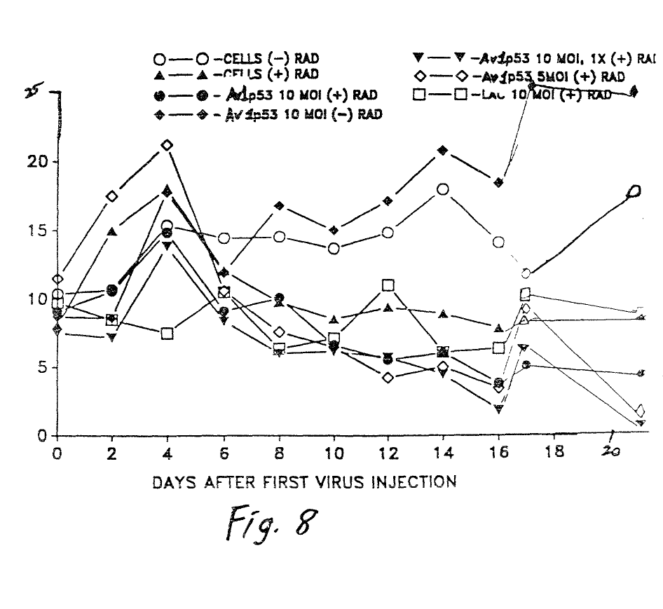

1×10⁶ JSQ-3 cells (in 50 ul PBS) were again injected subcutaneously on the lower back, above the tail, of 4-6 week-old female athymic nude mice. The results of this example are shown in FIG. 8. In brief, once the tumor has reached approximately 6-8 mm³ in volume, they were randomly divided into seven groups as described below:

Group 1. Control, without virus infection, without irradiation.

Group 2. LacZ vector, two injections at 10 MOI each, plus irradiation.

Group 3. X-irradiation, without virus injection.

Group 4. Av1p53, two injections, at 10 MOI each, without irradiation.

Group 5. Av1p53, two injections at 10 MOI each, plus irradiation.

Group 6. Av1p53, two injections at 5 MOI each, plus irradiation.

Group 7. Av1p53, one injection at 10 MOI each, plus irradiation.

Thirty-six to forty-eight hours after virus injection, the tumor site was exposed to 5 Gy of radiation for the first two fractions Subsequent irradiations were performed every 48 hours for six times to a total dose of 25 Gy. Animals were euthanized and tumors taken for histology prior to injection of virus, 36-48 hours after the first virus injection (prior to irradiation), and after completion of the course of radiation therapy. As a control, tumors were injected with the control viral vector (Av1LacZ4) at 10 MOI with irradiation. The tumor response, growth or inhibition, are monitored and measured as tumor volume using the equation: Cubic Volume=[L×(W)²]/2.

The experiment has entered its third week and the full course of radiation has been completed. A clear inhibitory effect of radiation on mean tumor volume is evident. All groups with radiation treatment have maintained the tumor volume either the same or smaller then the original volume when the first dose of adenovirus was injected. Tumors in the two groups (1 and 4) without irradiation increased in size exponentially entering the third week.

The disclosure of all patents, publications (including published patent applications), and database accession numbers and depository accession numbers referenced in this specification are specifically incorporated herein by reference in their entirety to the same extent as if each such individual patent, publication, and database accession numbers and depository accession numbers were specifically and individually indicated to be incorporated by reference.

It is to be understood, however, that the scope of the present invention is not to be limited to the specific embodiments described above. The invention may be practiced other than as particularly described and still be within the scope of the accompanying claims.

What is claimed is:

1. A process for improving the treatment of a tumor by radiation therapy comprising:
treating a tumor by radiation therapy wherein cells of said tumor have been transduced with a polynucleotide encoding wild type p53.

2. The process of claim 1 wherein said polynucleotide encoding wild-type p53 is contained within a viral vector.

3. The process of claim 2 wherein said viral vector is a DNA virus vector.

4. The process of claim 3 wherein said DNA virus vector is an adenoviral vector.

5. The process of claim 4 wherein said adenoviral vector is administered in an amount of from about 1×10⁷ pfu to about 1×10¹⁰ pfu.

6. The process of claim 5 wherein said adenoviral vector is administered in an amount of from about 5×10⁷ pfu to about 1×10⁹ pfu.

7. The process of claim 1 wherein said radiation is gamma radiation.

8. The process of claim 1 wherein said radiation is in the form of x-rays.

9. The process of claim 1 wherein said tumor is a squamous cell carcinoma of the head and neck.

10. The process of claim 1 wherein said radiation is administered in a total amount of from about 20 Gy to about 50 Gy.

11. The process of claim 10 wherein said radiation is administered in a total amount of from about 20 Gy to about 25 Gy.
